(19) United States
(12) Patent Application Publication
(10) Pub. No.: US 2024/0407670 A1
(43) Pub. Date: Dec. 12, 2024

(54) METHOD, DEVICE, AND RECORDING MEDIUM FOR ESTIMATING EXERCISE FUNCTION INDEX VALUE VARIATION, AND METHOD, DEVICE, AND RECORDING MEDIUM FOR GENERATING EXERCISE FUNCTION INDEX VALUE VARIATION ESTIMATION MODEL

(71) Applicants: PANASONIC HOLDINGS CORPORATION, Osaka (JP); POLARIS INC., Hyogo (JP)

(72) Inventors: Toshiaki TANAKA, Hyogo (JP); Maho Shiotani, Osaka (JP); Mika Saeki, Tokyo (JP)

(21) Appl. No.: 18/808,410

(22) Filed: Aug. 19, 2024

Related U.S. Application Data
(63) Continuation of application No. PCT/JP2023/005111, filed on Feb. 15, 2023.

(30) Foreign Application Priority Data
Feb. 21, 2022 (JP) 2022-024861

(51) Int. Cl. A61B 5/11; A61B 5/00; A61B 5/22; G16H 20/30

(52) U.S. Cl. CPC A61B 5/1124; A61B 5/224; A61B 5/4884; A61B 5/7267; A61B 5/7278; G16H 20/30; A61B 2503/08

(57) ABSTRACT

An information processing device acquires physical strength measurement data of a target; extracts a plurality of feature amounts based on the physical strength measurement data having been acquired; calculates a motor function index value variation of the target by inputting the plurality of feature amounts having been extracted to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and outputs the motor function index value variation having been calculated.

### FIELD OF INVENTION

The present disclosure relates to a method, a device, and a recording medium for estimating a motor function index value variation, and a method, a device, and a recording medium for generating a motor function index value variation estimation model.

### BACKGROUND ART

Non-Patent Literature 1 described below discloses a technique for estimating a TUG value of a target based on a measurement value of an acceleration sensor attached to the target.

Non-Patent Literature 1 includes no consideration on estimating the TUG value variation from the start of rehabilitation and after the passage of a predetermined period of time of a target.

Non-Patent Literature 1: Saporito Salvatore et al., “Remote timed up and go evaluation from activities of daily living reveals changing mobility”, Physiological Measurement, 2019

### SUMMARY OF THE INVENTION

An object of the present disclosure is to obtain a method, a device, and a program for estimating a motor function index value variation, and a method, a device, and a program for generating a motor function index value variation estimation model that can highly accurately estimate a motor function index value variation from the start of rehabilitation and after the passage of a predetermined period of time of a target.

In a method for estimating a motor function index value variation according to one aspect of the present disclosure, an information processing device acquires physical strength measurement data of a target, extracts a plurality of feature amounts based on the physical strength measurement data having been acquired, calculates a motor function index value variation of the target by inputting the plurality of feature amounts having been extracted to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects, and outputs the motor function index value variation having been calculated.

### BRIEF DESCRIPTION OF THE DRAWINGS

FIG. 1 is a diagram illustrating a simplified configuration of an information processing system according to an embodiment of the present disclosure.

FIG. 2 is a diagram illustrating a simplified configuration of a server device.

FIG. 3 is a flowchart showing generation processing of an estimation model executed by a processing unit of the server device in a learning phase.

FIG. 4 is a flowchart showing estimation processing of a TUG value variation executed by the processing unit of the server device in a use phase.

FIG. 5 is a graph showing estimation accuracy of the estimation model.

### DETAILED DESCRIPTION

### (Knowledge Underlying Present Disclosure)

In self-support nursing care facilities for elderly people and the like, deterioration in quality of nursing care services due to shortage of human resources and deterioration in productivity of nursing care staff, and non-uniformity in quality of nursing care services due to experience differences of nursing care staff and the like are problems on the site.

In order to improve and uniformize the quality of nursing care services, it is important to individually optimize the content of the nursing care service for each target, and in order to achieve the optimization, utilization of artificial intelligence is expected as a means for quantitatively evaluating the motor function of each target. By estimating an improvement amount in the motor function of each target in advance based on data that can be measured and input at home or the like before entering or visiting a facility, the facility side can grasp the improvement amount of the motor function of each target in advance, and as a result, it is possible to support individual optimization of the nursing care service. By presenting the improvement amount in the motor function from the facility side to each target, it is possible to encourage each target to enter or visit the facility.

One of indices used to evaluate the motor function of the elderly and the like is a timed up and go (TUG) value. Non-Patent Literature 1 discloses a technique including attaching an acceleration sensor to a target, remotely collecting a measurement value of the acceleration sensor within a predetermined period of time in which the target performs daily activities, and estimating a current TUG value of the target based on the collected measurement value.

However, Non-Patent Literature 1 includes no consideration on estimating the TUG value variation from the start of rehabilitation and after the passage of a predetermined period of time of a target. In generation of the estimation model, users having a good motor function in which an actual measurement value of the TUG value is about 5 to 10 seconds are targeted as subjects, and a feature amount necessary for construction of an estimation model for users (TUG value of about 15 seconds or more) requiring self-support nursing care is not considered.

In order to solve such a problem, by performing machine learning using physical strength measurement data or the like of each subject with users in need of the self-support nursing care as a subject, the present inventor has made clear a feature amount necessary for generation of an estimation model for users in need of self-support nursing care, has found that it is possible to construct a highly accurate motor function index value variation estimation model by using the feature amount, and has arrived at the present disclosure.

Next, each aspect of the present disclosure will be described.

A method for estimating a motor function index value variation according to a first aspect of the present disclosure includes: by an information processing device, acquiring physical strength measurement data of a target; extracting a plurality of feature amounts based on the physical strength measurement data having been acquired; calculating a motor function index value variation of the target by inputting the plurality of feature amounts having been extracted to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and outputting the motor function index value variation having been calculated.

According to the first aspect, the information processing device extracts a plurality of feature amounts based on the physical strength measurement data of the target, and calculates the motor function index value variation of the target by inputting the plurality of feature amounts having been extracted to the learned motor function index value variation estimation model. This enables the motor function index value variation from the start of rehabilitation and after the passage of a predetermined period of time of the target to be estimated with high accuracy.

In a method for estimating a motor function index value variation according to a second aspect of the present disclosure, in the first aspect, the motor function index value is a timed up and go (TUG) value.

According to the second aspect, the TUG value variation of the target can be estimated with high accuracy.

In a method for estimating a motor function index value variation according to a third aspect of the present disclosure, in the first or second aspect, the plurality of feature amounts include at least one of a test result of a TUG test and a test result of a one-leg stand test, which pertains to the target, as feature amounts based on the physical strength measurement data.

According to the third aspect, the estimation accuracy of the motor function index value variation can be improved.

In a method for estimating a motor function index value variation according to a fourth aspect of the present disclosure, in the third aspect, the test result of the TUG test includes time stability based on a difference value of a plurality of test results obtained from a plurality of TUG tests, and the test result of the one-leg stand test includes at least one of time stability based on a difference value of a plurality of test results obtained from a plurality of one-leg stand tests with a same leg of a left leg and a right leg and left-right stability based on a difference value of a plurality of test results obtained from a plurality of one-leg stand tests with different legs of a left leg and a right leg.

According to the fourth aspect, the estimation accuracy of the motor function index value variation can be further improved.

A method for estimating a motor function index value variation according to a fifth aspect of the present disclosure, in any one of the first to fourth aspects, includes: by the information processing device, further acquiring basic data including physical information of the target; and extracting the plurality of feature amounts based on the physical strength measurement data and the basic data having been acquired.

According to the fifth aspect, the estimation accuracy of the motor function index value variation can be improved.

In a method for estimating a motor function index value variation according to a sixth aspect of the present disclosure, in the fifth aspect, the basic data further includes disease information that pertains to the target.

According to the sixth aspect, the estimation accuracy of the motor function index value variation can be further improved.

In a method for estimating a motor function index value variation according to a seventh aspect of the present disclosure, in the fifth or sixth aspect, the plurality of feature amounts include, as feature amounts based on the basic data, at least one of physical information, disease information, walking ability information, drug-taking information, meal intake status information, fluid intake status information, care level information, daily life information, denture use status information, and rehabilitation implementation status information, which pertain to the target.

According to the seventh aspect, the estimation accuracy of the motor function index value variation can be further improved.

A device for estimating a motor function index value variation according to an eighth aspect of the present disclosure includes: an acquisition unit that acquires physical strength measurement data of a target; an extraction unit that extracts a plurality of feature amounts based on the physical strength measurement data having been acquired by the acquisition unit; a calculation unit that calculates a motor function index value variation of the target by inputting the plurality of feature amounts having been extracted by the extraction unit to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and an output unit that outputs the motor function index value variation having been calculated by the calculation unit.

According to the eighth aspect, the extraction unit extracts the plurality of feature amounts based on the physical strength measurement data of the target, and the calculation unit calculates the motor function index value variation of the target by inputting the plurality of feature amounts extracted by the extraction unit to the learned motor function index value variation estimation model. This enables the motor function index value variation from the start of rehabilitation and after the passage of a predetermined period of time of the target to be estimated with high accuracy.

A program for estimating a motor function index value variation according to a ninth aspect of the present disclosure is a program for causing an information processing device to function as: an acquisition means configured to acquire physical strength measurement data of a target; an extraction means configured to extract a plurality of feature amounts based on the physical strength measurement data having been acquired by the acquisition means; a calculation means configured to calculate a motor function index value variation of the target by inputting the plurality of feature amounts having been extracted by the extraction means to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and an output means configured to output the motor function index value variation having been calculated by the calculation means.

According to the ninth aspect, the extraction means extracts the plurality of feature amounts based on the physical strength measurement data of the target, and the calculation means calculates the motor function index value variation of the target by inputting the plurality of feature amounts extracted by the extraction means to the learned motor function index value variation estimation model. This enables the motor function index value variation from the start of rehabilitation and after the passage of a predetermined period of time of the target to be estimated with high accuracy.

A method for generating a motor function index value variation estimation model according to a tenth aspect of the present disclosure includes: by an information processing device, acquiring physical strength measurement data and actual measurement data of a motor function index value variation from start of rehabilitation and after passage of a predetermined period of time, which pertain to each of a plurality of subjects; extracting a plurality of feature amounts based on the physical strength measurement data having been acquired; and generating a motor function index value variation estimation model by machine learning using, as training data, the plurality of feature amounts and the actual measurement data, which pertain to each of a plurality of subjects.

According to the tenth aspect, the information processing device acquires the physical strength measurement data of each subject and the actual measurement data of the motor function index value variation from the start of rehabilitation and after the passage of a predetermined period of time, extracts a plurality of feature amounts based on the physical strength measurement data having been acquired, and generates the motor function index value variation estimation model by machine learning using, as training data, the plurality of feature amounts and the actual measurement data of each subject. This can generate a highly accurate motor function index value variation estimation model.

In a method for generating a motor function index value variation estimation model according to an eleventh aspect of the present disclosure, in the tenth aspect, the motor function index value is a timed up and go (TUG) value.

According to the eleventh aspect, it is possible to generate a highly accurate TUG value variation estimation model.

In a method for generating a motor function index value variation estimation model according to a twelfth aspect of the present disclosure, in the tenth or eleventh aspect, the plurality of feature amounts include at least one of a test result of a TUG test and a test result of a one-leg stand test, which pertains to each of the plurality of subjects, as feature amounts based on the physical strength measurement data.

According to the twelfth aspect, the estimation accuracy of the motor function index value variation estimation model can be improved.

In a method for generating a motor function index value variation estimation model according to a thirteenth aspect of the present disclosure, in the twelfth aspect, the test result of the TUG test includes time stability based on a difference value of a plurality of test results obtained from a plurality of TUG tests, and the test result of the one-leg stand test includes at least one of time stability based on a difference value of a plurality of test results obtained from a plurality of one-leg stand tests with a same leg of a left leg and a right leg and left-right stability based on a difference value of a plurality of test results obtained from a plurality of one-leg stand tests with different legs of a left leg and a right leg.

According to the thirteenth aspect, the estimation accuracy of the motor function index value variation estimation model can be further improved.

A method for generating a motor function index value variation estimation model according to a fourteenth aspect of the present disclosure, in any one of the tenth to thirteenth aspects, includes: by the information processing device, further acquiring basic data including physical information of each of the plurality of subjects; and extracting the plurality of feature amounts based on the physical strength measurement data and the basic data having been acquired.

According to the fourteenth aspect, the estimation accuracy of the motor function index value variation estimation model can be improved.

In a method for generating a motor function index value variation estimation model according to a fifteenth aspect of the present disclosure, in the fourteenth aspect, the basic data further includes disease information, which pertains to each of the plurality of subjects.

According to the fifteenth aspect, the estimation accuracy of the motor function index value variation estimation model can be further improved.

In a method for generating a motor function index value variation estimation model according to a sixteenth aspect of the present disclosure, in the fourteenth or fifteenth aspect, the plurality of feature amounts include, as feature amounts based on the basic data, at least one of physical information, disease information, walking ability information, drug-taking information, meal intake status information, fluid intake status information, care level information, daily life information, denture use status information, and rehabilitation implementation status information, which pertain to each of the plurality of subjects.

According to the sixteenth aspect, the estimation accuracy of the motor function index value variation estimation model can be further improved.

A device for estimating a motor function index value variation estimation model according to a seventeenth aspect of the present disclosure includes: an acquisition unit that acquires physical strength measurement data and actual measurement data of a motor function index value variation from start of rehabilitation and after passage of a predetermined period of time, which pertain to each of a plurality of subjects; an extraction unit that extracts a plurality of feature amounts based on the physical strength measurement data having been acquired by the acquisition unit; and a generation unit that generates a motor function index value variation estimation model by machine learning using, as training data, the plurality of feature amounts, which pertain to each of a plurality of subjects, extracted by the extraction unit, and the actual measurement data acquired by the acquisition unit.

According to the seventeenth aspect, the acquisition unit acquires the physical strength measurement data and the actual measurement data of the motor function index value variation from the start of rehabilitation and after the passage of a predetermined period of time of each subject, the extraction unit extracts the plurality of feature amounts based on the physical strength measurement data acquired by the acquisition unit, and the generation unit generates the motor function index value variation estimation model by machine learning using, as training data, the plurality of feature amounts and the actual measurement data of each subject. This can generate a highly accurate motor function index value variation estimation model.

A program for generating a motor function index value variation estimation model according to an eighteenth aspect of the present disclosure is a program for causing an information processing device to function as: an acquisition means configured to acquire physical strength measurement data and actual measurement data of a motor function index value variation from start of rehabilitation and after passage of a predetermined period of time, which pertain to each of a plurality of subjects; an extraction means configured to extract a plurality of feature amounts based on the physical strength measurement data having been acquired by the acquisition means; and a generation means configured to generate a motor function index value variation estimation model by machine learning using, as training data, the plurality of feature amounts extracted by the extraction means and the actual measurement data acquired by the acquisition unit, which pertain to each of a plurality of subjects.

According to the eighteenth aspect, the acquisition means acquires the physical strength measurement data and the actual measurement data of the motor function index value variation from the start of rehabilitation and after the passage of a predetermined period of time of each subject, the extraction means extracts the plurality of feature amounts based on the physical strength measurement data acquired by the acquisition means, and the generation means generates the motor function index value variation estimation model by machine learning using, as training data, the plurality of feature amounts and the actual measurement data of each subject. This can generate a highly accurate motor function index value variation estimation model.

An embodiment of the present disclosure will be described in detail below with reference to the drawings. Note that elements denoted by the same reference signs in different drawings represent the same or corresponding elements.

Note that each embodiment described below shows one specific example of the present disclosure. Numerical values, shapes, components, steps, order of steps, and the like shown in the following embodiment are merely examples, and are not intended to limit the present disclosure. A component that is not described in an independent claim representing the highest concept among components in the embodiment below is described as a discretionary component. In all the embodiments, respective items of content can be combined.

### Embodiments of Present Disclosure

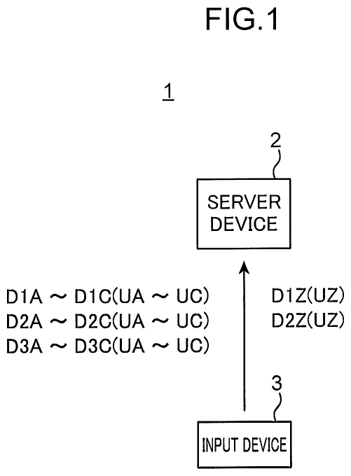

FIG. 1 is a diagram illustrating a simplified configuration of an information processing system 1 according to an embodiment of the present disclosure. The information processing system 1 includes a server device 2 and an input device 3.

The server device 2 is an edge server on the premises, a cloud server out of the premises, or the like. The server device 2 can wirelessly communicate with the input device 3 via a discretionary communication network.

The input device 3 is a PC, a tablet, a smartphone, or the like with which input operation is possible by the user or a staff member of a self-support nursing care facility.

The input device 3 transmits, to the server device 2, physical strength measurement data D1 (D1A to D1C and D1Z) indicating the result of a physical strength measurement test conducted on subjects UA to UC and a target UZ prior to start of rehabilitation in the self-support nursing care facility. The physical strength measurement test is a timed up and go (TUG) test and a one-leg stand test in the example of the present embodiment, but is not limited to this. The TUG test is a test for measuring the time required from the start of the standing-up motion until the subject again sits down in a series of motions in which the subject or the target stands up from a state of being seated in a chair and starts walking, turns back at a target point 3 m ahead, and sits down again in the chair. The one-leg stand test is a test for measuring the duration of one-leg standing by the subject or the target.

The subjects UA to UC are users who provide the physical strength measurement data D1 and the like in order to generate an estimation model 31 of the motor function index value variation described later. The motor function index value is a TUG value in the example of the present embodiment, but is not limited to this. For simplification of the drawing, FIG. 1 illustrates three subjects UA to UC, but the number of subjects is not limited to three as long as the number of subjects is plural. The target UZ is a user of a target for whom the TUG value variation from the start of rehabilitation and after the passage of a predetermined period of time using the estimation model 31 having been learned. The predetermined period of time is three months in the example of the present embodiment, but is not limited to this. The subjects UA to UC and the target UZ are elderly persons of users in need of self-support nursing care, for example. The target UZ is a user who is before entering or visiting a self-support nursing care facility, for example.

The input device 3 transmits, to the server device 2, basic data D2 (D2A to D2C and D2Z) that pertain to the subjects UA to UC and the target UZ, respectively. The content of the basic data D2 will be described later. The input device 3 transmits, to the server device 2, actual measurement data D3A to D3C of the TUG value indicating the result of a TUG test conducted for the respective subjects UA to UC from the start of rehabilitation and after the passage of a predetermined period of time.

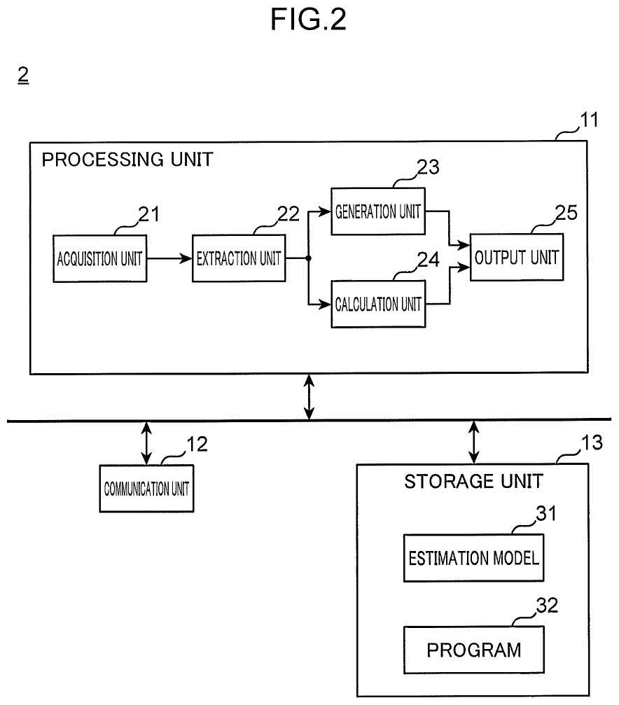

FIG. 2 is a diagram illustrating a simplified configuration of the server device 2. The server device 2 includes a processing unit 11, a communication unit 12, and a storage unit 13 connected to one another via a bus. The processing unit 11 is configured to include a processor such as a CPU. The communication unit 12 is configured to include a communication module compatible with a communication system with the input device 3. The storage unit 13 is configured to include an HDD, an SSD, or a semiconductor memory. The storage unit 13 stores an estimation model 31 having been learned. However, the estimation model 31 may be stored in an internal memory of not the storage unit 13 but the processing unit 11. The storage unit 13 stores a program 32.

As functions implemented by the processor executing the program 32 read from the storage unit 13, the processing unit 11 includes an acquisition unit 21, an extraction unit 22, a generation unit 23, a calculation unit 24, and an output unit 25. In other words, the program 32 is a program for generating the estimation model 31 for causing the server device 2 as an information processing device (device for generating estimation model) to function as the acquisition unit 21 (acquisition means), the extraction unit 22 (extraction means), the generation unit 23 (generation means), and the output unit 25 (output means) in a learning phase for generating the estimation model 31. The program 32 is a program for estimating a TUG value variation for causing the server device 2 as an information processing device (device for estimating TUG value variation) to function as the acquisition unit 21 (acquisition means), the extraction unit 22 (extraction means), the calculation unit 24 (calculation means), and the output unit 25 (output means) in a use phase of estimating the TUG value variation of the target UZ using the estimation model 31 having been learned.

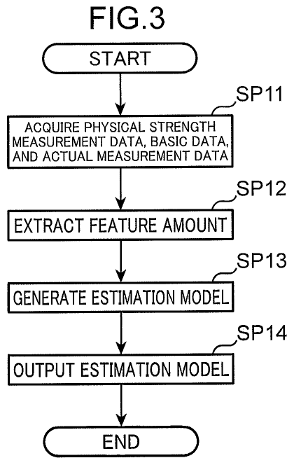

FIG. 3 is a flowchart showing the generation processing of the estimation model 31 executed by the processing unit 11 of the server device 2 in the learning phase.

First, in step SP11, the acquisition unit 21 acquires the physical strength measurement data D1A to D1C, the basic data D2A to D2C, and the actual measurement data D3A to D3C, which pertain to the respective subjects UA to UC.

In the example of the present embodiment, as the physical strength measurement test, the TUG test, the one-leg stand test with right-leg stand, and the one-leg stand test with left-leg stand are each conducted twice.

The physical strength measurement data D1 includes at least one of the following information.
test result of first TUG test
test result of second TUG test
best value of test results of two TUG tests
difference absolute value (time stability) of test results of two TUG tests
test result of first right one-leg stand test
test result of second right one-leg stand test
test result of first left one-leg stand test
test result of second left one-leg stand test
best value of test results of total of four one-leg stand tests
value (time stability) of larger one of difference absolute value of test results of two right one-leg stand tests and difference absolute value of test results of two left one-leg stand tests
value (left-right stability) of larger one of difference absolute value between test result of first right one-leg stand test and test result of first left one-leg stand test and difference absolute value between test result of second right one-leg stand test and test result of second left one-leg stand test

The basic data D2 includes at least one of physical information, disease information, walking ability information, drug-taking information, meal intake status information, fluid intake status information, care level information, daily life information, denture use status information, and rehabilitation implementation status information, which pertain to each of the plurality of subjects UA to UC prior to the start of rehabilitation.

The physical information includes at least one of the following information.
age, gender, height, weight, and BMI

The disease information includes at least one of the following information.
previous history of stroke, diabetes, heart disease, orthopedic disease, neurogenic progression disease (Parkinson's disease, etc.), dementia, sleep disorder, mental disease, cancer, or unknown disease
presence or absence of paralysis, severity of paralysis, symptom type of dementia, presence or absence of restlessness of dementia, and presence or absence of higher brain function disorder
severity of each disease, presence or absence of contracture, severity of contracture, presence or absence of treatment of contracture, presence or absence of walking disorder, type of walking disorder, and severity of walking disorder

The walking ability information includes at least one of the following information.
distance by which continuous walking is possible or practiced, time during which continuous walking is possible or practiced
status pertaining to self-support, walk while holding onto things, use of cane, use of walker, and use of wheelchair indoors
status pertaining to self-support, walk while holding onto things, use of cane, use of walker, and use of wheelchair outdoors

The drug-taking information includes at least one of the following information.
presence or absence of a laxative, presence or absence of a soporific, a type of a soporific, presence or absence of a diabetes drug, a type of a diabetes drug, presence or absence of a diuretic, and other drug-taking information

The meal intake status information includes at least one of the following information.
meal form, calorie intake, meal content, and PFC balance

The fluid intake status information includes at least one of the following information.
fluid intake amount and fluid intake timing

The care level information includes at least one of the following information.
certification type of business target, in need of support, and in need of nursing care

The daily life information includes at least one of the following information.
whether or not to be capable of daily activities such as rising, sitting, rolling over, standing up, and standing
living condition information such as regularity of life rhythm and going-out frequency

The denture use status information includes at least one of the following information.
presence or absence of denture adaptation and presence or absence of denture use

The rehabilitation implementation status information includes at least one of the following information.
rehabilitation implementation scheduled frequency, rehabilitation implementation scheduled time, rehabilitation implementation status in other facilities

Next, in step SP12, the extraction unit 22 extracts a plurality of feature amounts based on the physical strength measurement data D1 and the basic data D2 acquired by the acquisition unit 21 by using a discretionary feature amount selection algorithm.

The extraction unit 22 extracts, for example, the following information as the feature amount based on the physical strength measurement data D1.
test result of first TUG test
test result of second TUG test
best value of test results of two TUG tests
difference absolute value (time stability) of test results of two TUG tests
test result of first right one-leg stand test
test result of second right one-leg stand test
test result of second left one-leg stand test
best value of test results of total of four one-leg stand tests
value (time stability) of larger one of difference absolute value of test results of two right one-leg stand tests and difference absolute value of test results of two left one-leg stand tests
value (left-right stability) of larger one of difference absolute value between test result of first right one-leg stand test and test result of first left one-leg stand test and difference absolute value between test result of second right one-leg stand test and test result of second left one-leg stand test

The extraction unit 22 extracts, for example, the following information as the feature amount based on the basic data D2.
BMI
previous history of stroke, heart disease, neurogenic progression disease, mental disease, or unknown disease
whether or not to be capable of self-support, whether or not to be capable of walk while holding onto things, and presence or absence of use of wheelchair indoors
whether or not to be capable of self-support and presence or absence of use of walker outdoors
care level information

In the example of the present embodiment, the extraction unit 22 uses the K-Best method as the feature amount selection algorithm. However, in place of the K-Best method, an RFE method, a K-means method, Forward-Selection method, a brute-force method, or the like may be used.

Next, in step SP13, the generation unit 23 uses, as training data, the plurality of feature amounts extracted by the extraction unit 22 and the actual measurement data D3A to D3C acquired by the acquisition unit 21, and generates the estimation model 31 of the TUG value variation by machine learning using a discretionary model algorithm.

In the example of the present embodiment, the generation unit 23 uses an SVR model as a model algorithm.

As the model algorithm, a linear multiple regression model, SVM, XGBoostRegression, a neural network, a hierarchical Bayesian model, or the like may be used.

Next, in step SP14, the output unit 25 outputs the estimation model 31 generated by the generation unit 23, and stores the estimation model 31 in the storage unit 13. However, in a case where the estimation model 31 is stored in the internal memory of not the storage unit 13 but the processing unit 11, the output processing of the estimation model 31 by the output unit 25 is omitted.

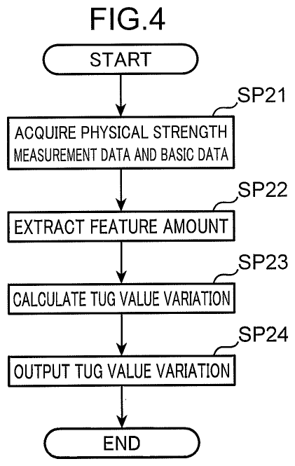

FIG. 4 is a flowchart showing the estimation processing of a TUG value variation executed by the processing unit 11 of the server device 2 in the use phase.

First, in step SP21, the acquisition unit 21 acquires the physical strength measurement data D1Z and the basic data D2Z pertaining to the target UZ.

Next, in step SP22, the extraction unit 22 extracts a plurality of feature amounts based on the physical strength measurement data D1Z and the basic data D2Z acquired by the acquisition unit 21 by using a discretionary feature amount selection algorithm. In the example of the present embodiment, the extraction unit 22 uses the K-Best method as the feature amount selection algorithm.

The extraction unit 22 extracts, for example, the following information as the feature amount based on the physical strength measurement data D1Z.
test result of first TUG test
test result of second TUG test
best value of test results of two TUG tests
difference absolute value (time stability) of test results of two TUG tests
test result of first right one-leg stand test
test result of second right one-leg stand test
test result of second left one-leg stand test
best value of test results of total of four one-leg stand tests
value (time stability) of larger one of difference absolute value of test results of two right one-leg stand tests and difference absolute value of test results of two left one-leg stand tests
value (left-right stability) of larger one of difference absolute value between test result of first right one-leg stand test and test result of first left one-leg stand test and difference absolute value between test result of second right one-leg stand test and test result of second left one-leg stand test

The extraction unit 22 extracts, for example, the following information as the feature amount based on the basic data D2Z.
BMI
previous history of stroke, heart disease, neurogenic progression disease, mental disease, or unknown disease
status pertaining to self-support, walk while holding onto things, and use of wheelchair indoors
status pertaining to self-support and use of walker outdoors
care level information

Next, in step SP23, the calculation unit 24 inputs the plurality of feature amounts extracted by the extraction unit 22 in step SP22 to the estimation model 31 having been learned generated in the learning phase to calculate the TUG value variation (estimation value) of the target UZ from the start of rehabilitation and after the passage of the predetermined period of time (e.g., 3 months).

Next, in step SP24, the output unit 25 outputs the TUG value variation of the target UZ calculated by the calculation unit 24.

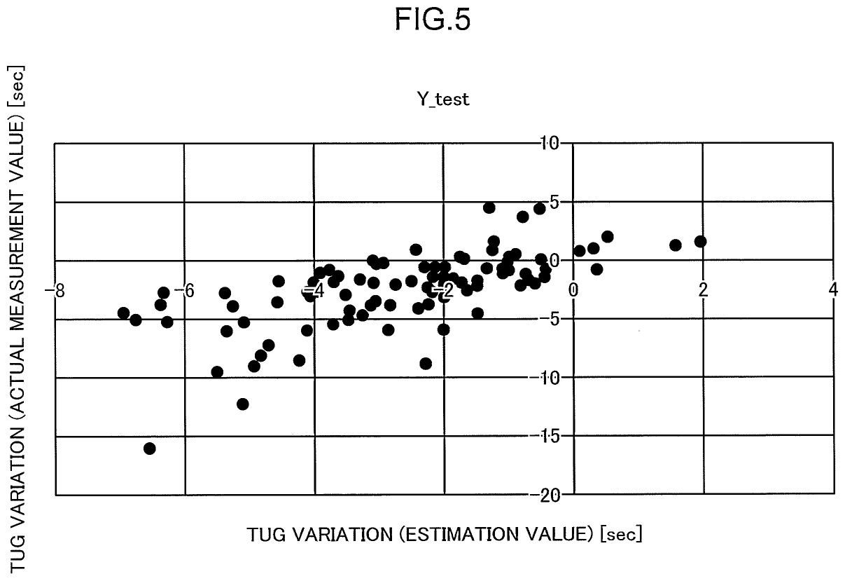

FIG. 5 is a graph showing estimation accuracy of the estimation model 31. Targeting users requiring self-support nursing care (TUG value before start of rehabilitation is about 15 seconds or more. However, users having a TUG value of 30 seconds or more are excluded), the estimation model 31 was generated by using the above 22 pieces of information as feature amounts. The vertical axis of the graph represents the actual measurement value of the TUG value variation, and the horizontal axis represents the estimation value of the TUG value variation using the estimation model 31. Although a large number of subjects having an actual measurement value of the TUG value variation of 15 seconds or more are included, the correlation coefficient exceeds 0.69, which suggesting that high estimation accuracy is obtained.

According to the present embodiment, the extraction unit 22 extracts a plurality of feature amounts based on the physical strength measurement data D1Z of the target UZ, and the calculation unit 24 calculates the TUG value variation of the target UZ by inputting the plurality of feature amounts extracted by the extraction unit 22 to the estimation model 31 of the TUG value variation having been learned. This enables the TUG value variation from the start of rehabilitation and after the passage of a predetermined period of time of the target UZ to be estimated with high accuracy.

In the above description, the extraction unit 22 extracts the plurality of feature amounts based on the physical strength measurement data D1 and the basic data D2, but it is sufficient to extract a plurality of feature amounts based on at least the physical strength measurement data D1, and the basic data D2 can be omitted.

In the above description, the basic data D2 includes the physical information, the disease information, the walking ability information, the drug-taking information, the meal intake status information, the fluid intake status information, the care level information, the daily life information, the denture use status information, and the rehabilitation implementation status information, but it is sufficient to include at least the physical information, it is sufficient to desirably include the physical information and the disease information, and other information can be omitted.

The physical strength measurement data D1 may include other measurement items as long as the items can be used to measure the motor ability of the target. For example, grip strength, walking speed, lower limb muscle strength, chair standing motion, 5-meter walking time, functional reach, and the like may be included.

The feature amount described above may further include sleep information, excretion information, physical condition information, inactivity level and period information, psychological/mental/motivation information, age/gender information, living alone/living together information, and external resource use information.

The disease information described above may further include a previous history, a symptom, cure, and treatment information.

The care level information described above may further include a care level, activities of daily living (ADL), and falling information such as the number of times/period of falling.

The meal intake status information described above may further include nutrition information such as an albumin value and denture information.

The basic data D2 may further include sleep information, excretion information, physical condition information, inactivity level and period information, psychological/mental/motivation information, age/gender information, living alone/living together information, and external resource use information.

The present disclosure is particularly useful for application to self-support nursing care AI for the purpose of improving and uniformizing the quality of nursing care services in self-support nursing care facilities.

1. A method for estimating a motor function index value variation, the method comprising:
by an information processing device,
acquiring physical strength measurement data of a target;
extracting a plurality of feature amounts based on the physical strength measurement data having been acquired;
calculating a motor function index value variation of the target by inputting the plurality of feature amounts having been extracted to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and
outputting the motor function index value variation having been calculated.

2. The method for estimating a motor function index value variation according to claim 1, wherein the motor function index value is a timed up and go (TUG) value.

3. The method for estimating a motor function index value variation according to claim 1, wherein the plurality of feature amounts include at least one of a test result of a TUG test and a test result of a one-leg stand test, which pertains to the target, as feature amounts based on the physical strength measurement data.

4. The method for estimating a motor function index value variation according to claim 3, wherein
the test result of the TUG test includes time stability based on a difference value of a plurality of test results obtained from a plurality of TUG tests, and
the test result of the one-leg stand test includes at least one of time stability based on a difference value of a plurality of test results obtained from a plurality of one-leg stand tests with a same leg of a left leg and a right leg and left-right stability based on a difference value of a plurality of test results obtained from a plurality of one-leg stand tests with different legs of a left leg and a right leg.

5. The method for estimating a motor function index value variation according to claim 1, comprising:
by the information processing device,
further acquiring basic data including physical information of the target; and
extracting the plurality of feature amounts based on the physical strength measurement data and the basic data having been acquired.

6. The method for estimating a motor function index value variation according to claim 5, wherein the basic data further includes disease information that pertains to the target.

7. The method for estimating a motor function index value variation according to claim 5, wherein the plurality of feature amounts include, as feature amounts based on the basic data, at least one of physical information, disease information, walking ability information, drug-taking information, meal intake status information, fluid intake status information, care level information, daily life information, denture use status information, and rehabilitation implementation status information, which pertain to the target.

8. A device for estimating a motor function index value variation, the device comprising:
an acquisition unit that acquires physical strength measurement data of a target;
an extraction unit that extracts a plurality of feature amounts based on the physical strength measurement data having been acquired by the acquisition unit;
a calculation unit that calculates a motor function index value variation of the target by inputting the plurality of feature amounts having been extracted by the extraction unit to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and
an output unit that outputs the motor function index value variation having been calculated by the calculation unit.

9. A computer-readable non-transitory recording medium recording a program for estimating a motor function index value variation, the program for causing an information processing device to perform a process comprising:
acquiring physical strength measurement data of a target;
extracting a plurality of feature amounts based on the physical strength measurement data having been acquired;
calculating a motor function index value variation of the target by inputting the plurality of feature amounts having been extracted to a learned motor function index value variation estimation model obtained by machine learning using, as training data, the plurality of feature amounts and actual measurement data of the motor function index value variation from start of rehabilitation and after passage of a predetermined period of time, which pertain to each of a plurality of subjects; and
outputting the motor function index value variation having been calculated by the calculation means.

10. A method for generating a motor function index value variation estimation model, the method comprising:
by an information processing device,
acquiring physical strength measurement data and actual measurement data of a motor function index value variation from start of rehabilitation and after passage of a predetermined period of time, which pertain to each of a plurality of subjects;
extracting a plurality of feature amounts based on the physical strength measurement data having been acquired; and
generating a motor function index value variation estimation model by machine learning using, as training data, the plurality of feature amounts and the actual measurement data, which pertain to each of a plurality of subjects.

11. The method for generating a motor function index value variation estimation model according to claim 10, wherein the motor function index value is a timed up and go (TUG) value.

12. The method for generating a motor function index value variation estimation model according to claim 10, wherein the plurality of feature amounts include at least one of a test result of a TUG test and a test result of a one-leg stand test, which pertains to each of the plurality of subjects, as feature amounts based on the physical strength measurement data.

13. The method for generating a motor function index value variation estimation model according to claim 12, wherein
the test result of the TUG test includes time stability based on a difference value of a plurality of test results obtained from a plurality of TUG tests, and
the test result of the one-leg stand test includes at least one of time stability based on a difference value of a plurality of test results obtained from a plurality of one-leg stand tests with a same leg of a left leg and a right leg and left-right stability based on a difference value of a plurality of test results obtained from a plurality of one-leg stand tests with different legs of a left leg and a right leg.

14. The method for generating a motor function index value variation estimation model according to claim 10, comprising:
by the information processing device,
further acquiring basic data including physical information of each of the plurality of subjects; and
extracting the plurality of feature amounts based on the physical strength measurement data and the basic data having been acquired.

15. The method for generating a motor function index value variation estimation model according to claim 14, wherein the basic data further includes disease information, which pertains to each of the plurality of subjects.

16. The method for generating a motor function index value variation estimation model according to claim 14, wherein the plurality of feature amounts include, as feature amounts based on the basic data, at least one of physical information, disease information, walking ability information, drug-taking information, meal intake status information, fluid intake status information, care level information, daily life information, denture use status information, and rehabilitation implementation status information, which pertain to each of the plurality of subjects.

17. A device for generating a motor function index value variation estimation model, the device comprising:
an acquisition unit that acquires physical strength measurement data and actual measurement data of a motor function index value variation from start of rehabilitation and after passage of a predetermined period of time, which pertain to each of a plurality of subjects;
an extraction unit that extracts a plurality of feature amounts based on the physical strength measurement data having been acquired by the acquisition unit; and
a generation unit that generates a motor function index value variation estimation model by machine learning using, as training data, the plurality of feature amounts, which pertain to each of a plurality of subjects, extracted by the extraction unit, and the actual measurement data acquired by the acquisition unit.

18. A computer-readable non-transitory recording medium recording a program for generating a motor function index value variation estimation model, the program for causing an information processing device to perform a process comprising:
acquiring physical strength measurement data and actual measurement data of a motor function index value variation from start of rehabilitation and after passage of a predetermined period of time, which pertain to each of a plurality of subjects;
extracting a plurality of feature amounts based on the physical strength measurement data having been acquired; and
generating a motor function index value variation estimation model by machine learning using, as training data, the plurality of feature amounts extracted and the actual measurement data acquired, which pertain to each of a plurality of subjects.
